SEQUENCE LISTING (13 sequences)

SEQ ID NO: 1 - DNA, 19 bp, synthetic construct
  source 1..19: mol_type = other DNA; note = primer
ggggcaatcgtatgcttct

SEQ ID NO: 2 - DNA, 19 bp, synthetic construct
  source 1..19: mol_type = other DNA; note = primer
cgggtatttgtctcggtgt

SEQ ID NO: 3 - DNA, 18 bp, synthetic construct
  source 1..18: mol_type = other DNA; note = primer
gtttgtgatgggcgtgaa

SEQ ID NO: 4 - DNA, 18 bp, synthetic construct
  source 1..18: mol_type = other DNA; note = primer
ctgttgctgtagccgaat

SEQ ID NO: 5 - DNA, 19 bp, synthetic construct
  source 1..19: mol_type = other DNA; note = primer
cttgtgcctcttagtcctg

SEQ ID NO: 6 - DNA, 18 bp, synthetic construct
  source 1..18: mol_type = other DNA; note = primer
acatagaatgccctggtg

SEQ ID NO: 7 - DNA, 20 bp, synthetic construct
  source 1..20: mol_type = other DNA; note = primer
atttgctggcgtattgagtg

SEQ ID NO: 8 - DNA, 20 bp, synthetic construct
  source 1..20: mol_type = other DNA; note = primer
aagacggtaggttctgtggg

SEQ ID NO: 9 - DNA, 22 bp, synthetic construct
  source 1..22: mol_type = other DNA; note = primer
tcataggccatagctaaatggt

SEQ ID NO: 10 - DNA, 20 bp, synthetic construct
  source 1..20: mol_type = other DNA; note = primer
gagacaggctgaatgtgcaa

SEQ ID NO: 11 - DNA, 22 bp, synthetic construct
  source 1..22: mol_type = other DNA; note = primer
tcataggccatagctaaatggt

SEQ ID NO: 12 - DNA, 20 bp, synthetic construct
  source 1..20: mol_type = other DNA; note = primer
gagacaggctgaatgtgcaa

SEQ ID NO: 13 - DNA, 271 bp, synthetic construct
  source 1..271: mol_type = other DNA; note = probe
tcataggccatagctaaatggtacttagagaaatttttatagatttaaatacattcatcagactacaaatgaagtgtaggcattaaaattaagaaacttgaagaatgtgtatgtacttccaaagcaaaaataagaaaaataagggtataactcgcttctagggactcaggggcagtctaattttcaagcactgagaagcccagattagtctaatcagtgcaagcctgtctttcagtccatttaaagatcctttgcacattcagcctgtctc

(19) United States
(12) Patent Application Publication
(10) Pub. No.: US 2023/0265506 A1
(43) Pub. Date: Aug. 24, 2023

(54) PIS GENOTYPE DETECTION METHOD FOR GOATS

(71) Applicant: QINGDAO AGRICULTURAL UNIVERSITY, QINGDAO (CN)

(72) Inventors: HEGANG LI, QINGDAO (CN); MENGMENG DU, QINGDAO (CN); XIAOKUN LIN, QINGDAO (CN); QUANWEI ZHANG, QINGDAO (CN); MING CHENG, QINGDAO (CN); JINSHAN ZHAO, QINGDAO (CN)

(73) Assignee: QINGDAO AGRICULTURAL UNIVERSITY, QINGDAO (CN)

(21) Appl. No.: 18/076,447

(22) Filed: Dec. 7, 2022

(30) Foreign Application Priority Data
Jun. 7, 2022 (CN) 202210638902.2

(51) Int. Cl. C12Q 1/6879

(52) U.S. Cl. CPC C12Q 1/6879; C12Q 2600/124; C12Q 2600/156

(57) ABSTRACT

The present invention discloses a PIS genotype detection method for goats and belongs to field of life science. The method includes 4 pairs of specific primers: SRY F and SRY R; PIS wt F and PIS wt R; PIS var-1 F and PIS var-1 R; and PIS var-2 F and PIS var-2 R, and a probe. The present invention further provides a specific detection method and a detection standard. A simple and efficient detection method for diagnosing a PIS genotype of the goats is developed in combination with a high-sensitivity PCR method and a high-stability Southern Blot method, has extremely high specificity, is convenient and rapid, and may be used for accurately identifying genotypes of PIS genes, guiding breeding and mating of the goats and controlling or adjusting genotype frequencies of the PIS genes in goat populations according to requirements of breeders, to increase economic benefits of goat farming.

### REFERENCE TO SEQUENCE LISTING

The substitute sequence listing is submitted as a XML file filed via EFS-Web, with a file name of “Substitute_Sequence_Listing_SJDL-U.S. Pat. No. 1,224,491-97-1810.XML”, a creation date of May 12, 2023, and a size of 14998 bytes. The substitute sequence Listing filed via EFS-Web is a part of the specification and is incorporated in its entirety by reference herein.

### CROSS REFERENCE TO RELATED APPLICATION

This patent application claims the benefit and priority of Chinese Patent Application No. 202210638902.2 filed on Jun. 7, 2022, the disclosure of which is incorporated by reference herein in its entirety as part of the present application.

### TECHNICAL FIELD

The present invention relates to the technical field of life science, and particularly relates to a PIS genotype detection method for goats.

### BACKGROUND

Horn is one of the signature characteristics of a goat, rather than an economic character. In modern animal husbandry, horned goats fight obviously and occupy a position of prominence, may scramble for feed during feeding, and even cause conditions such as abortion, fracture and breast tearing during fighting. However, compared with the horned goats, polled goats decrease economic losses brought by fighting among individuals, and are favorable for large-scale captive breeding of livestock, thereby facilitating farming and management. Therefore, breeding a polled goat strain has become an important direction of the goat breeding field.

Intersex is a phenomenon that certain hermaphrodite animals have bisexual characters. Intersex of goats refers to female sex reverse in close relation to polled characters, and is also called polled intersex syndrome (PIS). Such a complex causal mutation is considered as mutation on Chromosome 1 of goats reported previously. The complex causal mutation also occurs in domestic breeds such as Laoshan dairy goats, Tangshan dairy goats and Jintang black goats. Thus, it is indicated that, great defects exist in early methods for detecting PIS genotype of goats; and the methods often take a part for the whole, are fruitless approaches, and fail to grasp the main points.

The intersex characters of the goats are caused by recessive mutation of a PIS region of Chromosome 1. Therefore, during breeding of the goats, if a PIS heterozygous mutant is not eliminated by virtue of some biological means, an intersex gene frequency of an offspring group is higher and higher. Meanwhile, an occurrence rate of intersex goats is increased, thereby bringing serious economic losses to goat farming industry. At present, existing intersex goat detection methods include a karyotype analysis method, PCR and the like. Since the karyotype analysis method is complex in operation and high in difficulty and cannot detect minute deficiency and other lesions, precise location of chromosome variation points is difficult to be provided. The PCR detection method has defects of low reliability, low primer specificity, low amplification efficiency and low stability.

In addition, fluorescence in-situ hybridization can detect structural variation of PIS chromosome by using hybridization between a specific probe and a chromosome in metaphase of cell division. However, due to huge workloads and higher cost, generally the fluorescence in-situ hybridization is less adopted.

Therefore, a problem that urgently needs to be solved by those skilled in the art is to develop a simple and efficient detection method for diagnosing a PIS genotype of the goats in combination with a high-sensitivity PCR method and a high-stability Southern Blot method.

### SUMMARY

In view of this, the present invention provides a PIS genotype detection method for goats. The detection method may be used for accurately identifying genotypes of PIS genes, guiding breeding and mating of the goats and controlling or adjusting genotype frequencies of PIS genes in goat populations according to requirements of breeders to increase economic benefits of goat farming.

To achieve the above purpose, technical solutions of the present invention are as follows:

A primer and a probe group for detecting PIS genotype of goats include 4 pairs of specific primers: SRY F and SRY R; PIS wt F and PIS wt R; PIS var-1 F and PIS var-1 R; and PIS var-2 F and PIS var-2 R, and a probe.

SRY F:
5′-GGGGCAATCGTATGCTTCT-3′,
shown as SEQ ID No. 1;
SRY R:
5′-CGGGTATTTGTCTCGGTGT-3′,
shown as SEQ ID No. 2;
PIS wt F:
5′-CTTGTGCCTCTTAGTCCTG-3′,
shown as SEQ ID No. 5;
PIS wt R:
5′-ACATAGAATGCCCTGGTG-3′,
shown as SEQ ID No. 6;
PIS var-1 F:
5′-ATTTGCTGGCGTATTGAGTG-3′,
shown as SEQ ID No. 7;
PIS var-1 R:
5′-AAGACGGTAGGTTCTGTGGG-3′,
shown as SEQ ID No. 8;
PIS var-2 F:
5′-TCATAGGCCATAGCTAAATGGT-3′,
shown as SEQ ID No. 9;
PIS var-2 R:
5′-GAGACAGGCTGAATGTGCAA-3′,
shown as SEQ ID No. 10;
the probe:
5′-TCATAGGCCATAGCTAAATGGTACTTAGAGA
AATTTTTATAGATTTAAATACATTCATCAGACTA
CAAATGAAGTGTAGGCATTAAAATTAAGAAACTT
GAAGAATGTGTATGTACTTCCAAAGCAAAAATAA
GAAAAATAAGGGTATAACTCGCTTCTAGGGACTC
AGGGGCAGTCTAATTTTCAAGCACTGAGAAGCCC
AGATTAGTCTAATCAGTGCAAGCCTGTCTTTCAG
TCCATTTAAAGATCCTTTGCACATTCAGCCTGTC
TC-3′,
shown as SEQ ID No. 11.

Preferably, the primer and the probe group further include a specific primer pair of GAPDH F and GAPDH R;

GAPDH F:
5′-GTTTGTGATGGGCGTGAA-3′,
shown as SEQ ID No. 3;
GAPDH R:
5′-CTGTTGCTGTAGCCGAAT-4′,
shown as SEQ ID No. 4.

The present invention further provides a kit based on the above detection primer and probe group.

The present invention further provides an application of the above detection primer and probe group or the above kit in livestock breeding.

The present invention further provides a PIS genotype detection method for goats based on the above detection primer and probe group. The method includes the following steps:

(1) conducting primer design: including genetic sex determination primers and PIS genotype identification primers;

(2) conducting genetic sex and PIS genotype identification; and

(3) preparing a probe and conducting Southern hybridization detection.

Preferably, the step (2) includes: extraction of sample genome DNA; PCR amplification of the genetic sex determination primers SRY-F/R and GAPDH-F/R; PCR amplification of the PIS genotype identification primers PIS wt F/R and PIS var-1 F/R; a 25 μL system is adopted during amplification and includes 22 μL of 1.1×Golden Star Super PCR Mix, 1 μL of template DNA, 1 μL of forward primers and 1 μL of reverse primers; and reaction procedures are as follows: conducting pre-denaturation at 95° C. for 5 min, conducting denaturation at 95° C. for 30 s, annealing for 30 s, extending at 72° C. for 30 s, conducting cycles for 34 times, conducting renaturation at 72° C. for 5 min, and preserving the product at 4° C.

Preferably, the step (3) includes: sample extraction; PIS var-2 F/R primer synthesis; recovery and purification of a target fragment; clone sequencing; extraction of a recombinant plasmid; preparation of a probe; labeling of the probe; digestion of genome DNA; and electrophoresis, membrane transfer, hybridization, membrane cleaning and signal detection on a digestion product.

PIS var-2 F/R primer synthesis: a 25 μL system is adopted and includes 22 μL of 1.1×Golden Star Super PCR Mix, 1 μL of template DNA, 1 μL of forward primers and 1 μL of reverse primers; and PCR reaction procedures are as follows: conducting pre-denaturation at 95° C. for 5 min, conducting denaturation at 95° C. for 30 s, annealing at 60° C. for 30 s, extending at 72° C. for 30 s, conducting cycles for 34 times, conducting renaturation at 72° C. for 5 min, and preserving the product at 4° C.

Clone sequencing: the carrier is a pGM-T carrier; a reaction system includes 1 μL of 10×T4 DNA Ligation Buffer, 1 μL of 3 U/μL T4 DNA Ligase, 1 μL of a 50 ng/μL pGM-T carrier and 7 μL of a target PCR fragment; and overnight ligation is conducted at 16° C.

Preparation of the probe: the probe is synthesized through PCR by taking a recombinant plasmid as a template; labeled mononucleotide is doped into the newly synthesized DNA strands in the presence of Taq polymerase; a 50 μL system is adopted during PCR amplification and includes 5 μL of plus Mg²⁺10×buffer, 5 μL of a dUTP labeled mixture, 1 μL of forward primers, 1 μL of reverse primers, 1 μL of the Taq polymerase, 1 μL of the recombinant plasmid and 36 μL of ddH₂O; and PCR reaction procedures are as follows: conducting pre-denaturation at 95° C. for 5 min, conducting denaturation at 95° C. for 30 s, annealing at 55° C. for 30 s, extending at 72° C. for 20 s, conducting cycles for 35 times, conducting renaturation at 72° C. for 7 min, and preserving the product at 4° C.

Digestion of genome DNA: a 800 μL system is adopted and includes 60 μL of 10×buffer, 15 μL of BgI II, 15 μL of EcoR I, 30 μL of genome DNA and 680 μL of ddH₂O.

Preferably, detection standards are as follows:

Genetic Sex:

horned ewes: the amplification product has only one band of 579 bp;

horned rams: the amplification product has two bands of 579 bp and 379 bp simultaneously;

polled ewes: the amplification product has only one band of 579 bp;

polled rams: the amplification product has two bands of 579 bp and 379 bp simultaneously;

intersex goats: the amplification product has only one band of 579 bp; and the chromosome is XX;

PIS Typing:

wild-type homozygous horned goats: the amplification product has only one fragment of 214 bp;

deletion-mutation homozygous intersex goats: the amplification product has only one fragment of 680 bp, and there is a band during Southern hybridization detection;

dual-deletion homozygous polled rams: the amplification product has only one fragment of 680 bp, and there is a band during Southern hybridization detection;

deletion-mutation homozygous polled goats: the amplification product has fragments of 214 bp and 680 bp simultaneously, and there is a band during Southern hybridization detection.

Through the above technical solutions, compared with the prior art, the present invention discloses and provides the PIS genotype detection method for goats. Technical effects are achieved as follows: problems such as low reliability, low primer specificity, low amplification efficiency and low stability existing in the current PCR detection method for the PIS genotype of the goats are solved. In addition, fluorescence in-situ hybridization realizes hybridization between a specific probe and a chromosome in metaphase of cell division. However, a PIS region is mainly composed of repetitive sequences; preparation of a DNA probe suitable for hybridization is relatively difficult; and the probe is often prepared by a BAC cloning method. Moreover, the steps are various and complicated; signal loss is easily caused; a technical obstacle of false negative results is caused.

Further, a simple and efficient detection method for diagnosing the PIS genotype of the goats is first developed in combination with the high-sensitivity PCR method and the high-stability Southern Blot method, has extremely high specificity, is convenient and rapid, and may be used for accurately identifying genotypes of PIS genes, guiding breeding and mating of the goats and controlling or adjusting genotype frequencies of the PIS genes in goat populations according to requirements of breeders, to increase economic benefits of goat farming.

### DETAILED DESCRIPTION OF THE DRAWINGS

To more clearly describe the technical solutions in the embodiments of the present invention or in the prior art, the drawings required to be used in the description of the embodiments or the prior art will be simply presented below. Apparently, the drawings in the following description are merely the embodiments of the present invention, and for those ordinary skilled in the art, other drawings can also be obtained according to the provided drawings without contributing creative labor.

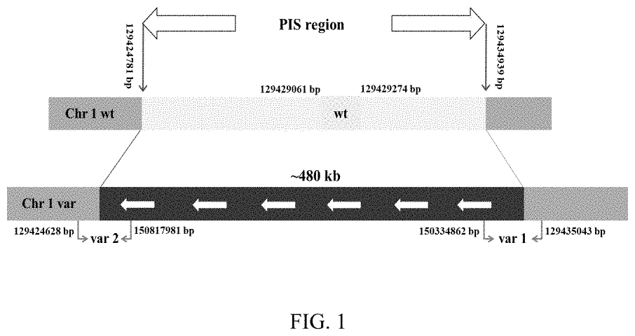

FIG. 1 is a schematic diagram of location of PIS genotype identification primers in Chromosome 1 provided by the present invention;

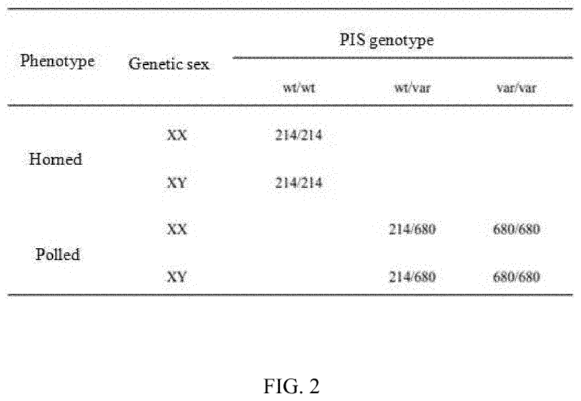

FIG. 2 is a schematic diagram of sizes of amplification fragments of three PIS reproductive deficiency genotypes provided by the present invention;

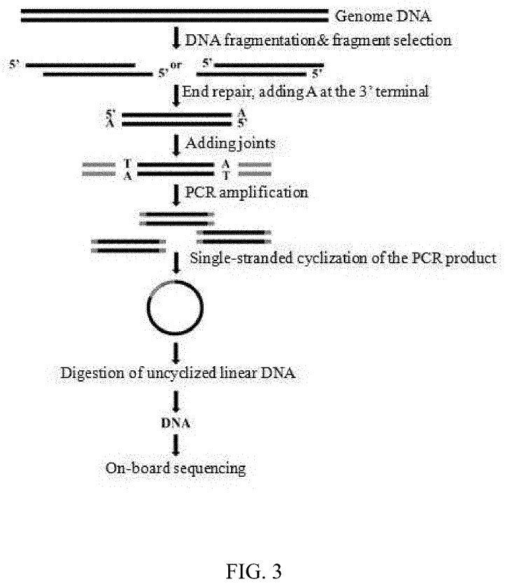

FIG. 3 is a schematic diagram of a library construction sequencing process provided by the present invention;

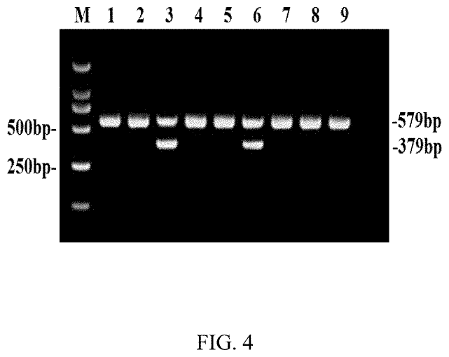

FIG. 4 is a genetic sex determination result map provided by the present invention, wherein M: DL 2000 DNA Marker; 1-2: representative samples of horned ewes; 3: representative sample of horned ram; 4-5: representative samples of polled ewes; 6: representative sample of polled ram; 7-9: representative samples of intersex goats;

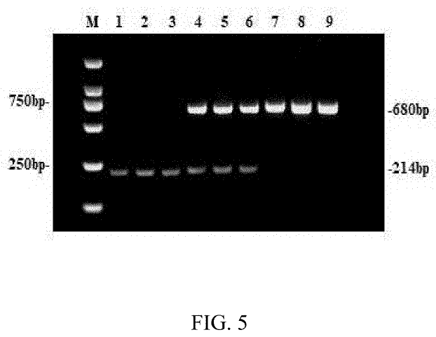

FIG. 5 is an electrophoretogram of three genotypes of PIS reproductive deficiency genes of Laoshan dairy goats provided by the present invention, wherein M: DL 2000 DNA Marker; 1-3: representative samples of horned goats; 4-6: representative samples of polled goats; 7-9: representative samples of intersex goats;

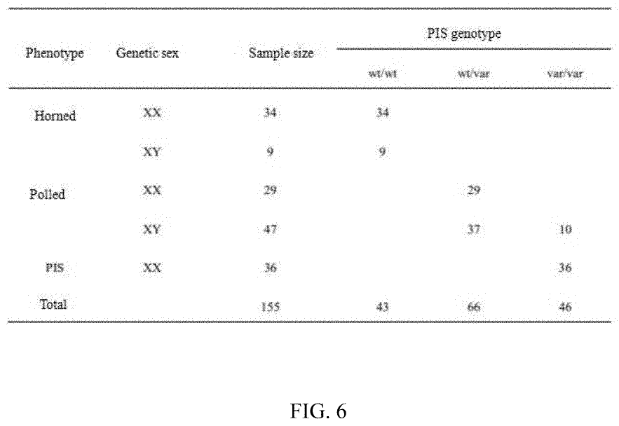

FIG. 6 is a statistical chart of detection results of PIS reproductive deficiency genotypes of Laoshan dairy goat populations provided by the present invention;

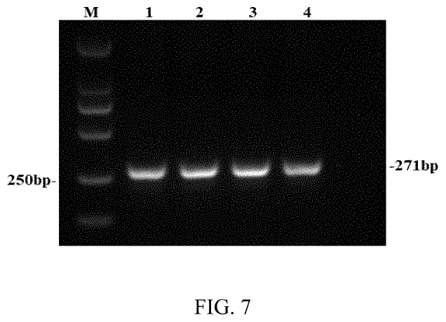

FIG. 7 is a PCR amplification result map of probe primers provided by the present invention, wherein M: DL 2000 DNA Marker; 1-4: gel electrophoresis after PCR amplification of the probe primers;

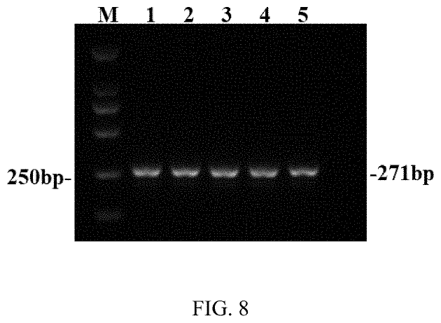

FIG. 8 is a PCR detection result map of a bacterium solution provided by the present invention, wherein M: DL 2000 DNA Marker; 1-5: PCR gel electrophoresis detection results of single colonies;

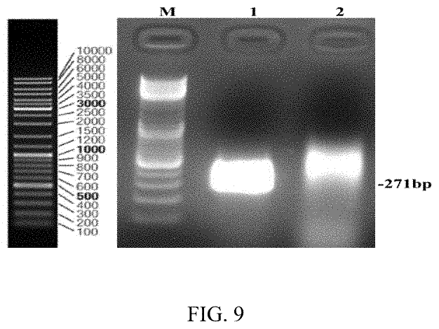

FIG. 9 is an electrophoresis result map of a labeled probe provided by the present invention, wherein M: DL 10000 DNA Marker; 1: DNA fragment of the control; 2: labeled DNA probe;

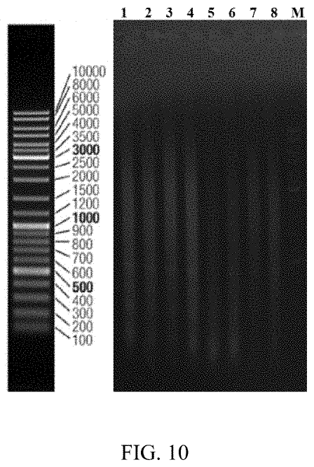

FIG. 10 is a DNA double-digestion electrophoresis result map provided by the present invention, wherein M: DL 10000 DNA Marker; 1-2: DNA digestion results of horned goats; 3-5: DNA digestion results of polled goats; 6-8: DNA digestion results of intersex goats;

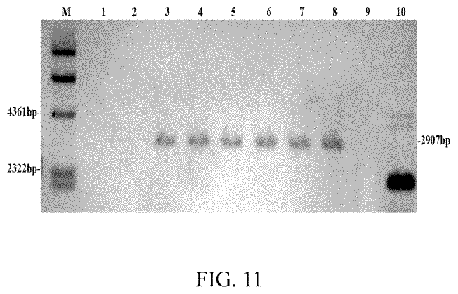

FIG. 11 is a Southern hybridization result map provided by the present invention, wherein M: DNA Molecular-Weight Marker II; 1-2: horned goats; 3-5: polled goats; 6-8: intersex goats; 9: negative control; and 10: positive control; and

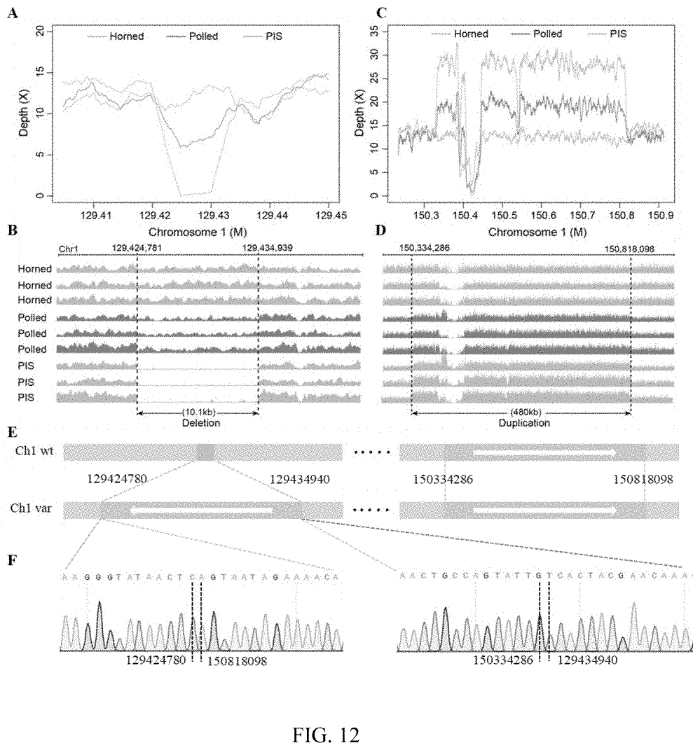

FIG. 12 is a structural variation characteristic pattern provided by the present invention, wherein A: genome coverage rate of 10.1 kb deletion; B: aligned short sequence screenshot of 10.1 kb deletion on Chromosome 1; C: genome coverage rate of 480 kb duplication; D: aligned short sequence screenshot of 480 kb duplication on Chromosome 1; E: inverted insertion description figure of a 480 kb duplication sequence on a PIS locus; and F: PCR sequencing result of structural variation.

### DETAILED DESCRIPTION OF THE EMBODIMENTS

The technical solutions in the embodiments of the present invention will be clearly and fully described below in combination with the drawings in the embodiments of the present invention.

Apparently, the described embodiments are merely part of the embodiments of the present invention, not all of the embodiments. Based on the embodiments in the present invention, all other embodiments obtained by those ordinary skilled in the art without contributing creative labor will belong to the protection scope of the present invention.

Embodiments of the present invention disclose a PIS genotype detection method for goats.

Principles:

1. Establishment of PCR Detection Method

(1) Primer design: primers involved in the present invention include two parts: genetic sex determination primers and PIS genotype identification primers.

Design of the genetic sex determination primers refers to a SRY sequence (JN561348), and internal reference primers refer to a GAPDH sequence (NM_001190390). The primers are designed by utilizing Primer Premier 5.0 software.

The PIS genotype identification primers are analyzed and designed according to 10.1 kb deletion/480 kb duplication characteristics of a PIS region on Chromosome 1 of Saanen dairy goats in combination with complete sequence (RefSeq: NC_030808.1) sequence of goat genome DNA released by NCBI. A wild-type detection primer PIS wt is designed in a 10.1 kb deletion region of Chromosome 1, and has an amplification fragment size of 214 bp. A variation detection primer PIS var-1 is respectively designed at each of two ends of downstream breakpoints, and has an amplification fragment size of 680 bp. Pre-experiment detection is conducted on the designed primers, to ensure that the primers have higher specificity.

(2) Genetic sex determination: SRY and GAPDH gene fragments are amplified by utilizing a PCR technology; and the genetic sex of the detected individual is determined by detecting existence of the 2 specific fragments. If the two specific fragments (370 bp and 579 bp) exist simultaneously, the individual is male; and if only the fragment of 579 bp exists, the individual is female.

(3) PIS reproductive deficiency genotype identification: the identification of the PIS reproductive deficiency genotype totally involves 2 pairs of primers; PIS wt and PIS var-1 gene fragments are amplified by utilizing the PCR technology; and PIS genotypes of 155 goats are determined by detecting the existence of the 2 specific fragments.

2. Establishment of Southern Hybridization Detection Method

(1) Preparation of probe: a primer PIS var-2 is respectively designed at each of two ends of upstream breakpoints; a specific location of the primer is shown as FIG. 1; a PCR product is recovered and purified and ligated onto a pGM-T carrier for clone sequencing; a positive bacterium solution is selected for extracting a plasmid; and the probe is synthesized through PCR by taking the plasmid as a template.

(2) Digestion of genome DNA: digestion sites are designed according to the complete sequence (RefSeq: NC_030808.1) information of the goat genome DNA. The genome DNA is subjected to double digestion by selecting restriction enzymes BgI II and EcoR I. Before double digestion, a pair of primers including the double digestion sites is respectively designed, goat genome DNA of 3 dairy goats is subjected to PCR amplification and sequencing, and then SnapGene software is used to analyze whether a sequencing sequence contains the corresponding digestion sites, to ensure that no change of the digestion sites is caused due to interspecific or individual difference.

(3) Southern Hybridization Detection

After the digestion results are detected through electrophoresis, overnight exposure is conducted in a darkroom after membrane transfer, fixation, hybridization, washing and signal detection, and the results are recorded. According to PCR detection results, the intersex goats have 2 variation copy numbers, and there is a band during hybridization; the polled goats have 1 variation copy number, and there is a band during hybridization; and the horned goats are of the wild type without mutation, and there is no band during hybridization.

3. Validation of genome-wide re-sequencing (only further validation of the PCR and Southern detection results, for proving reliability of the method in the present patent from a genome-wide level).

Genome DNA of blood of 3 horned goats, 3 polled goats and 3 intersex goats is extracted; the DNA is randomly broken by utilizing Covaris ultrasonic waves; DNA fragments of 200-300 bp are recovered; the joints are added; and a library with ligated joints was subjected to linear amplification (LM-PCR). An appropriate amount of the amplification product is subjected to single strand separation and cyclization treatment; the cyclization library is subjected to rolling circle amplification (RCA) to generate a DNA nanoball (DNB); and the DNA nanoball is subjected to on-board sequencing after qualification. The sequencing results are compared with that of the reference goat genome; and structural variation characteristics of the Chromosome 1 of the polled and intersex goats are analyzed.

As shown in FIG. 1, the PIS genotype identification primers are designed according to the 10.1 kb deletion/480 kb duplication characteristics of the PIS region on Chromosome 1 of the goats in combination with the complete sequence (RefSeq: NC_030808.1) information of goat genome DNA released by NCBI. Genotypes and genetic rules of PIS reproductive deficiency genes of Laoshan dairy goats are analyzed by utilizing the PCR technology; and amplification fragment combinations of the goats of different genotypes are shown as FIG. 2. Specific primers (primer sequences are shown as Table 1, and the probe sequences are shown as Table 2) and corresponding double digestion sites are designed at two ends of the upstream breakpoint of the 10.1 kb deletion/480 kb duplication of the PIS region; the Southern hybridization detection method is established; and the PIS genotypes of the Laoshan dairy goats of different characters are further analyzed.

As shown in FIG. 3, 3 horned goats, 3 polled goats and 3 intersex goats are subjected to genome-wide re-sequencing. Through comparison with the reference goat genome, genetic variation data related to polled characters and intersex characters are obtained, thereby further analyzing mutation characteristics of the PIS region of the Laoshan dairy goats.

TABLE 1
Primer sequence information

| | | Product | Annealing |
|---|---|---|---|
| Primer | | length | temperature |
| name | Primer sequence () | (bp) | (° C.) |
| SRY | F: GGGGCAATCGTATGCTTCT, shown as | | |
| | SEQ ID No. 1 | 379 | 48 |
| GAPDH | R: CGGGTATTTGTCTCGGTGT, shown as | | |
| | SEQ ID No. 2 | | |
| | F: GTTTGTGATGGGCGTGAA, shown as | | |
| | SEQ ID No. 3 | 579 | 43 |
| | R: CTGTTGCTGTAGCCGAAT, shown as | | |
| | SEQ ID No. 4 | | |
| PIS wt | F: CTTGTGCCTCTTAGTCCTG, shown as | 214 | 62 |
| | SEQ ID No. 5 | | |
| | R: ACATAGAATGCCCTGGTG, shown as | | |
| | SEQ ID No. 6 | | |
| PIS var-1 | F: ATTTGCTGGCGTATTGAGTG, shown as | 680 | 66 |
| | SEQ ID No. 7 | | |
| | R: AAGACGGTAGGTTCTGTGGG, shown | | |
| | as SEQ ID No. 8 | | |
| PIS var-2 | F: TCATAGGCCATAGCTAAATGGT, shown | 271 | 60 |
| | as SEQ ID No. 9 | | |
| | R: GAGACAGGCTGAATGTGCAA, shown | | |
| | as SEQ ID No. 10 | | |

TABLE 2
Probe sequence information

tcataggccatagctaaatggtacttagagaaatt
tttatagatttaaatacattcatcagactacaaat
gaagtgtaggcattaaaattaagaaacttgaagaa
tgtgtatgtacttccaaagcaaaaataagaaaaat
aagggtataactcgcttctagggactcaggggcag
tctaattttcaagcactgagaagcccagattagtc
taatcagtgcaagcctgtctttcagtccatttaaa
gatcctttgcacattcagcctgtctc,
shown as SEQ ID No. 11;

### Embodiment 1

1. Test Sample

Whole blood samples of 155 Laoshan dairy goats were collected in Qingdao area, including 36 intersex goats, 9 horned rams, 34 horned ewes, 29 polled ewes and 47 polled rams. 10 mL of jugular vein blood was collected and immediately cryopreserved in liquid nitrogen.

2. Extraction of Genome DNA

An adsorption column was placed in a collection tube; 250 μL of Buffer BL was added; centrifugation was conducted at 12000 rpm for 1 min; and a silica membrane was activated. 20 μL of Protrinase K was added into a 1.5 mL centrifuge tube. 200 μL of the fully and uniformly mixed whole blood samples were added and then fully and uniformly shaken and mixed. 200 μL of Buffer qAl was added, fully and uniformly shaken and mixed, and incubated at 70° C. for 15 min; and shaking was conducted for 3-5 times during the period. 200 μL of Ethanol was added after completion of incubation, and fully and uniformly shaken and mixed. The mixed solution obtained in the last step was totally added into the adsorption column and centrifuged at 12000 rpm for 2 min; and the solution was removed. 500 μL of Buffer PW diluted with Ethanol was added into the adsorption column and centrifuged at 12000 rpm for 2 min; the solution was removed; and the operation was repeated once. 500 μL of Wash Buffer diluted with Ethanol was added into the adsorption column and centrifuged at 12000 rpm for 1 min; and the solution was removed. The adsorption column was placed back in the collection tube; idling centrifugation was conducted at 14000 rpm for 2 min; the solution was removed; and the adsorption column was aired at room temperature for 5 min. The adsorption column was put in a 1.5 mL sterile enzyme-free centrifuge tube; 80 μL of TE Buffer preheated at 65° C. was added to the center of the adsorption column; the adsorption column was stood at 25° C. for 5 min; and centrifugation was conducted at 14000 rpm for 2 min. The product was preserved at −20° C.

3. Amplification of Genetic Sex Determination Primers

A 25 μL system was adopted during PCR amplification and included 22 μL of 1.1×Golden Star Super PCR Mix, 1 μL of template DNA, 1 μL of a forward primer and 1 μL of a reverse primer (SRY-F/R and GAPDH-F/R). PCR reaction procedures were as follows: pre-denaturation was conducted at 95° C. for 5 min, denaturation was conducted at 95° C. for 30 s, annealing was conducted for 30 s, extension was conducted at 72° C. for 30 s, cycles were conducted for 34 times, renaturation was conducted at 72° C. for 5 min, and the product was preserved at 4° C. A PCR product was detected through 2% agarose gel electrophoresis. Detection results were shown as FIG. 4.

The results showed that, in the 34 horned ewes, amplification results of 2 samples were shown as Lane 1-2 (amplification results of the rest horned ewe samples were shown as Lane 1-2, similarly hereinafter), and the amplification product had only one band of 579 bp; Lane 3 represented the 9 horned rams, and the amplification product had two bands of 579 bp and 379 bp simultaneously; Lane 4-5 represented the 29 polled ewes, and the amplification product had only one band of 579 bp; Lane 6 represented the 47 polled rams, and the amplification product had two bands of 579 bp and 379 bp simultaneously; and Lane 7-9 represented the 36 intersex rams, and the amplification product had only one band of 579 bp, which indicated that the sex chromosome of the 36 intersex rams was XX.

4. Amplification of PIS Reproductive Deficiency Genotype Identification Primers

PIS reproductive deficiency genotype identification was the same as the genetic sex determination. PIS genotype detection results of Laoshan dairy goat populations were shown as FIG. 5 and FIG. 6. Lane 1-3 had a fragment of 214 bp; and representative samples indicated that the genotype of the 43 horned rams was wild-type homozygote, and 10.1 kb deletion/480 kb duplication did not occur. Only a fragment of 680 bp was detected on Lane 7-9; and representative samples indicated that the genotype of the 36 intersex goats was deletion-mutation homozygote, and dual deletion of 10.1 kb and dual duplication of 480 kb occurred simultaneously. Lane 4-6 represented 76 normal polled goats, wherein 10 goats were dual-deletion homozygous polled rams (having the fragment of 680 bp only); the rest deletion-mutation homozygous normal polled goats had the fragment of 214 bp and the fragment of 680 bp simultaneously and had the genotype of mutation homozygote; and mutant genes included 10.1 kb deletion/480 kb duplication. Such a result indicated that, occurrence of the intersex goats was caused by complete insertion of the 10.1 kb and a reversely inserted repeat fragment of about 480 kb. Moreover, the intersex characters occurred in homozygous ewes only, while the intersex characters did not occur in homozygous deletion of the rams.

### Embodiment 2

Establishment of Southern Hybridization Detection Method for PIS Mutation of Laoshan Dairy Goats

1. Test Samples

3 intersex goats, 3 polled goats and 2 horned goats used in the present test were selected from Laoyikang Laoshan dairy goat stock seed farm in Laoshan district, Qingdao. PCR detection results of PIS genotypes of the goats were respectively 10.1 kb deletion/480 kb duplication combined mutation, 10.1 kb deletion/480 kb duplication single mutation and wild type without 10.1 kb deletion/480 kb duplication. Ear tissues of each goat were cut by ear forceps, disinfected with 75% of alcohol and then added into a 1.5 mL sterilized centrifuge tube; and the centrifuge tube was rapidly put in liquid nitrogen for cryopreservation.

2. Synthesis of Probe Primer

A 25 μL system was adopted during PCR amplification and included 22 μL of 1.1×Golden Star Super PCR Mix, 1 μL of template DNA (intersex goat), 1 μL of a forward primer and 1 μL of a reverse primer (PIS var-2). PCR reaction procedures were as follows: pre-denaturation was conducted at 95° C. for 5 min, denaturation was conducted at 95° C. for 30 s, annealing was conducted at 60° C. for 30 s, extension was conducted at 72° C. for 30 s, cycles were conducted for 34 times, renaturation was conducted at 72° C. for 5 min, and the product was preserved at 4° C. A PCR product was detected through 2% agarose gel electrophoresis. Results were shown as FIG. 7: Lane 1-4 were all the electrophoresis detection results of the PCR product; and the band was single, was in line with an expected band of 271 bp, and may be used for later clone sequencing.

3. Recovery and Purification of Target Fragment

A centrifuge tube filled with a glue block was placed in an analytical balance for weighing to obtain net weight; a volume of the glue block was calculated according to 1 mg=1 μL after weighing completion; and the glue block was cut up. After the glue block was cut up, dissolution of the glue block may be accelerated; and a recovery rate of DNA was increased. An addition amount of Buffer GM was calculated according to the weight of the glue block and the concentration of gel. When the concentration of gel was 2%, every 1 mg of the glue block corresponded to 5 μL of Buffer GM. A uniform mixture of the glue block and the Buffer GM was added into a water bath of 37° C.; and the water bath was slightly shaken to fully dissolve the mixture until the solution was transparent. An adsorption column in the kit was assembled. The glue block mixed solution that was completely dissolved in the last step was transferred into the adsorption column; centrifugation was conducted at 12000 rpm for 1 min to increase the recovery rate of the DNA; and the filtrate may be added into the adsorption column for further centrifugation. 700 μL of Buffer WB was added into the center of the adsorption column after completion of centrifugation; centrifugation was conducted at 12000 rpm at 25° C. for 1 min; the solution was removed; and the operation was repeated once. Idling centrifugation was conducted at 14000 rpm at 25° C. for 2 min. After completion of centrifugation, the adsorption column was put into a 1.5 mL sterile enzyme-free centrifuge tube and stood at 25° C. for 5 min. After completion of standing, 30 μL of Elution Buffer preheated at 60° C. was added to the center of the adsorption column, and the adsorption column was stood at 25° C. for 2 min. Then, centrifugation was conducted at 14000 rpm at 25° C. for 2 min. To increase DNA purity, further centrifugation may be conducted. The product was preserved at −20° C.

4. Clone Sequencing

A pGM-T carrier was melted on ice and subjected to transient centrifugation. 1 μL of 10×T4 DNA Ligation Buffer, 1 μL of T4 DNA Ligase (3 U/μL), 1 μL of the pGM-T carrier (50 ng/μL) and 7 L of a target PCR fragment were sequentially added into a sterile centrifuge tube. The centrifuge tube was slightly flipped to mix the components; transient centrifugation was conducted; and overnight ligation was conducted at 16° C. 5 μL of the ligation product was added into 50 μL of TOP 10 competent cells; the ligation product and the competent cells were slightly blown and beaten for uniform mixing; and the mixture was placed in an ice bath for 30 min.

After completion of the ice bath, the mixture was immediately placed in a water bath at 42° C. for heat shock for 90 s; the mixture was immediately placed in an ice bath for 10 min after completion of the heat shock; and the centrifuge tube cannot be shaken during the period. After completion of the ice bath, 250 μL of antibiotic-free fluid LB was added into the mixture; and the mixture was cultured at 200 rpm at 37° C. for 1 h. After the bacterium solution was blown, beaten and uniformly mixed, 150 L of the bacterium solution was inoculated onto an ampicillin-containing solid medium; and the solid medium was cultured overnight in a thermostatic incubator at 37° C. The obtained white single colony was inoculated into a 1.5 mL centrifuge tube containing 1 mL of LB (containing 50 mg/mL of ampicillin); and the centrifuge tube was shaken and cultured for 3-4 h in a shaker at 37° C. After completion of culture, PCR identification was conducted on the bacterium solution by utilizing the primer PIS var-2. The amplification system and reaction procedures were the same as those in synthesis of the probe primer. Identification results were shown as FIG. 8: Lane 1-5 represented 5 selected single colonies; and electrophoretic bands were all single and bright, were all about 271 bp and were in line with expected results. The selected colonies 1, 3 and 5 were sent to the company for sequencing; and plasmids were extracted according to sequencing results.

5. Extraction of Recombinant Plasmid

100 μL of positive bacterium solution was added into 15 mL of fluid LB broth containing 30 μL of Amp; the solution was shaken and cultured overnight in a shaker at 37° C.; 5 mL of the bacterium solution was added into a 1.5 mL centrifuge tube for multiple times; centrifugation was conducted at room temperature at 10000 rpm for 1 min; the supernatant was removed; 250 μL of Solution I (containing RNase A) was added; the solution was blown and beaten from top to bottom by a pipette for uniform mixing; 250 μL of Solution II was added, slightly inverted and uniformly mixed and incubated at room temperature for 2 min. A Buffer N3 solution was pre-cooled in an ice block in advance; 125 μL of Buffer N3 was added into the above centrifuge tube and immediately inverted and uniformly mixed until a white flocculent precipitate appeared, and centrifugation was conducted at 12000 rpm at 4° C. for 10 min. The supernatant was carefully sucked and added into the 1.5 mL sterile enzyme-free centrifuge tube; ETR Solution in a volume of 0.1 time was added, inverted and mixed to obtain turbid lysate; the lysate was incubated on ice for 10 min; and the solution was inverted and uniformly mixed for 3-5 times during incubation. After completion of the ice bath, the lysate was incubated in a water bath at 42° C. for 5 min; and centrifugation was conducted at 12000 rpm at room temperature for 2 min. The supernatant was transferred into a new 1.5 mL centrifuge tube; and Ethanol in a volume of 0.5 time was added, inverted and uniformly mixed, and incubated at room temperature for 2 min. An absorption column in the kit was mounted; 700 μL of the mixture in the last step was transferred into a 2 mL absorption column; and centrifugation was conducted at 10000 rpm at room temperature for 1 min. The filtrate was removed; the residual mixture was added into the absorption column; and centrifugation was conducted at 10000 rpm at room temperature for 1 min. The filtrate was removed; and the absorption column was reutilized. 500 μL of Buffer HBC was added; and centrifugation was conducted at 10000 rpm for 1 min. The absorption column was cleaned; and residual proteins were removed for ensuring DNA purity. The filtrate was removed; 700 μL of Wash Buffer diluted with Ethanol was added; and centrifugation was conducted at 10000 rpm at room temperature for 1 min. The filtrate was removed; and another 700 μL of the Wash Buffer was added for cleaning the absorption column. The filtrate was removed; an empty column was centrifuged at 13000 rpm for 2 min; and residual alcohol was removed. The absorption column was put into a new 1.5 mL centrifuge tube; 30-50 μL of Elution Buffer was added into the center of a filter membrane; the filter membrane was stood for 2 min; and centrifugation was conducted at 13000 rpm for 1 min. The product was preserved at −20° C.

6. Preparation of Probe

A probe was synthesized through PCR by taking a plasmid as a template. In the process, a primer sequence was PIS var-2-F-TCATAGGCCATAGCTAAATGGT, shown as SEQ ID No.9, or R-GAGACAGGCTGAATGTGCAA, shown as SEQ ID No.10. In the presence of Taq polymerase, labeled mononucleotide was doped into a newly synthesized DNA strand, so that a molecular weight of the labeled DNA fragment was increased. A 50 μL system was adopted during PCR amplification and included 5 μL of 10×buffer (plus Mg²⁺), 5 μL of a dUTP labeled mixture, 1 μL of forward primers, 1 μL of reverse primers, 1 μL of the Taq polymerase, 1 μL of a plasmid and 36 μL of ddH₂O. PCR reaction procedures were as follows: pre-denaturation was conducted at 95° C. for 5 min, denaturation was conducted at 95° C. for 30 s, annealing was conducted at 55° C. for 30 s, extension was conducted at 72° C. for 20 s, cycles were conducted for 35 times, renaturation was conducted at 72° C. for 7 min, and the product was preserved at 4° C. The PCR product was detected through 1% agarose gel electrophoresis. Detection results were shown as FIG. 9: the unlabeled electrophoretic band 1 had a length of 271 bp; the molecular weight of the labeled electrophoretic band 2 was increased; and the electrophoretic band 2 lagged behind the unlabeled electrophoretic band 1, which proved that the probe had been successfully labeled.

7. Labeling of the Probe

A DNA probe was labeled by random priming as follows: a 1 μg of template DNA probe was added into a PCR tube; then nuclease-free water was added to reach a volume of 16 μL; denaturation was conducted at 100° C. for 10 min; the tube was immediately put in an ice bath for 5 min after completion of denaturation; after DIG-High Prime blowing and uniform mixing, 4 μL of the denatured DNA was blown, beaten and uniformly mixed and then subjected to transient centrifugation; the reaction solution in the last step was incubated overnight at 37° C.; and finally, the solution was incubated at 65° C. for 10 min, and the reaction terminated.

8. Digestion of Genome DNA

A 800 μL system was adopted during digestion and included 60 μL of 10×buffer, 15 μL of BgI II, 15 μL of EcoR I, 30 μL of genome DNA and 680 μL of ddH₂O. An incision enzyme was added in three times; 10 μL of the incision enzyme was added for the first time, slightly blown, beaten and uniformly mixed and then digested at 37° C. for 2 h; then another 10 μL of the incision enzyme was added, slightly blown, beaten and uniformly mixed and then digested overnight at 37° C. (for about 16 h); and on the next day, another 10 μL of the incision enzyme was added for continuous reaction for 4 h. After completion of the reaction, 5 μL of the product was subjected to gel electrophoresis detection.

9. Agarose Gel Electrophoresis of Genome DNA

The digested genome DNA was subjected to 0.7% agarose gel electrophoresis to detect whether the genome DNA was completely digested. Electrophoresis conditions included: a constant voltage of 25V, a low temperature, overnight. Detection results were shown as FIG. 10: Lane 1-2 represented horned goats; Lane 3-5 represented polled goats; Lane 6-8 represented intersex goats; all electrophoretic bands were dispersive, which indicated that the digestion was complete, and a next membrane transfer test may be conducted.

10. Membrane Transfer and Fixation

A nylon membrane and filter paper were cut according to the size of glue. The gel was transferred into an incubation box; the incubation box was cleaned with sterilized distilled water twice; a denatured reaction solution in a volume of 5 times was added and shaken at room temperature for 45 min. The denatured reaction solution was poured; the incubation box was cleaned with the sterilized distilled water twice; a neutralizing reaction solution in a volume of 5 times was added and shaken at room temperature for 30 min. The neutralizing reaction solution was poured; the incubation box was cleaned with the sterilized distilled water twice; and 2×SSC in a volume of 5 times was added for soaking for 5 min. Then, the nylon membrane and the filter paper were soaked with the sterilized distilled water for 10 min; and the 2×SSC was added for soaking for 5 min. Overnight membrane transfer was conducted by an upward capillary method; the membrane was taken down and labeled after membrane transfer; and the membrane was washed with the 2×SSC for 5 min and then dried at 120° C. for 30 min.

11. Hybridization and Washing

10 mL of Hyb-100 was taken and added into a hybridization tube for prehybridization at 37° C. for 2 h. The prepared probe was denatured at 100° C. for 10 min and then immediately put in an ice bath to be cooled for 5 min; a prehybridization solution was poured; 20 μL of a newly denatured probe was added into the Hyb-100, blown, beaten and uniformly mixed and then hybridized overnight at 37° C.; the membrane was put in a new incubation box; 20 mL of 2×SSC/0.1% SDS was added and moderately shaken and washed at room temperature for 2×5 min. The membrane was transferred into an incubation box containing 20 mL of 1×SSC/0.1% SDS and then washed at 68° C. for 3×15 min.

12. Signal Detection

20 mL of Washing buffer was added for washing for 5 min. 10 mL of Blocking solution was added for incubating for 30 min. 10 mL of Antibody solution was added for incubating for 30 min. The Antibody solution was poured, and 20 mL of Washing buffer was added for slow washing for 2×15 min. 10 mL of Detection solution was added for balancing for 2-5 min. One side with DNA was upwards placed on a development folder, and 0.1 mL of CSPD was dropped onto the membrane; the membrane was sealed and incubated at room temperature for 5 min; and the membrane was exposed and shot on an X-ray film at room temperature. Results were shown as FIG. 11: Lane 1-2 represented horned goats; Lane 3-5 represented polled goats; Lane 6-8 represented intersex goats; Lane 9 represented negative control; Lane 10 represented positive control (plasmid); there was no band during hybridization of the horned goats, and the genotype of the horned goats was wild-type homozygote without mutation; and there were bands during hybridization of the polled and intersex goats, which was consistent with the PCR detection result.

### Embodiment 3

Analysis of Mutation Characteristics in PIS Region of Laoshan Dairy Goats by Utilizing Genome-Wide Re-Sequencing Technology

1. Construction of DNA Library

Integrity of sample genome DNA was detected through agarose gel electrophoresis; then concentrations of all samples were detected by utilizing a microplate reader; and qualified DNA samples were selected for next library construction. The qualified genome DNA samples were randomly broken into fragments of specific sizes by Covaris ultrasonic waves. The fragments of the broken samples were selected by Agencourt AMPure XP-Medium kit, so that sample bands were concentrated at about 200-300 bp; the amount of the purified DNA samples was detected through 500 assays by using a Qubit dsDNA HS AssayKit; a reaction system was prepared; an optimum temperature reaction was conducted for a certain time; terminals of double-stranded cDNA were repaired; base A was added at the terminal 3′; a joint ligation reaction system was prepared; and an optimum temperature reaction was conducted for a certain time, so that a sequencing joint was ligated with the DNA. A PCR reaction system was prepared; reaction procedures were set; and the ligation product was amplified. Fragments of the amplification product were screened by using a reagent Agencourt AMPure XP-Medium. The PCR product was detected by Agilent 2100 Bioanalyzer. After the PCR product was denatured into a single strand, a cyclization reaction system was prepared; the product was fully and uniformly mixed; an optimum temperature reaction was conducted for a certain time to obtain a single-stranded cyclic product; and after uncyclized linear DNA molecules were digested, a final library was obtained. Fragment sizes and concentrations of the library were detected by using the Agilent 2100 Bioanalyzer (Agilent DNA 1000 Reagents). The qualified library was subjected to on-board sequencing.

2. Filtering of Sequencing Data

Sequenced raw reads may carry adaptor, or contain a small amount of low-quality sequences.

Therefore, to ensure accuracy of subsequent analysis results, a series of data processing is needed for removing impurity data so as to obtain valid data. The analysis step is completed by using SOAPnuke (SOAPnuke 1.5.6) filter software; and filtering steps are as follows:

(1) filtering joints: deleting the entire read if sequenced read is matched with more than 50% of the adapter sequence;

(2) filtering low-quality data: deleting the entire read if bases with biased quality value in the sequenced read account for a certain ratio of the entire read; and

(3) removing N: deleting the entire read if N content in the sequenced read accounts for a certain ratio or more of the entire read.

3. Comparison

CleanData obtained after filtering is compared onto reference genome by using a “mem-t 10-M-Y” algorithm of BWABWA comparison software of short sequences to generate a comparison result file sam. Then, the file sam is converted into a sorted file bam by using a sort tool of samtools software. The comparison result file is further processed by utilizing the samtools software, e.g., the comparison file is subjected to sorting and quality control by utilizing the samtools software. Reads having mapQ values greater than 30 are selected for subsequent variation detection analysis.

Comparison Steps are as Follows:

(1) indexing according to the reference genome;

(2) comparing the reads with the reference genome, and searching compared coordinate information; and

(3) converting the compared coordinate information into a file bam by using the samtools software.

After completion of comparison, the file bam is sorted according to coordinates on the genome by using the samtools software; the comparison results are counted by using Qualimap2; and reads having comparison quality values smaller than 30 are filtered by using the samtools software.

Thus, the reads at multiple compared locations may be removed.

4. Structural Variation Detection

When structural variation exists in a sequencing sample sequence and a reference sequence, normal double-ended comparison data may be inadequate to meet detection conditions of the structural variation. Therefore, reads in abnormal comparison are further needed for participating in the structural variation detection. SV is detected by using BreakDancer. Detection results are shown as FIG. 12: in sequencing depth analysis on a 1 kb sliding window, there is about 10 kb deletion (FIG. 12-A) on Chromosome 1 of the polled and intersex goats; WGS data is detected by using Integrative Genomics Viewer (IGV); a breakpoint appears at 129 424 781-129 434 939 bp, and an exact length of the deletion is 10 159 bp (FIG. 12-B); and another variation length is about 480 kb, and the variation site is located at 150 334 286-150 818 098 bp (FIGS. 12-C and 12-D). In addition, the IGV and Sanger sequencing shows that, a 480 kb duplication sequence is reversely inserted into the genome location of the 10.1 kb deletion (FIGS. 12-E and 12-F).

Each embodiment in the description is described in a progressive way. The difference of each embodiment from each other is the focus of explanation. The same and similar parts among all of the embodiments can be referred to each other.

The above description of the disclosed embodiments enables those skilled in the art to realize or use the present invention. Many modifications to these embodiments will be apparent to those skilled in the art. The general principle defined herein can be realized in other embodiments without departing from the spirit or scope of the present invention. Therefore, the present invention will not be limited to these embodiments shown herein, but will conform to the widest scope consistent with the principle and novel features disclosed herein.

What is claimed is:

1. A primer and probe group for detecting PIS genotype of goats, comprising: 4 pairs of specific primers: SRY F and SRY R; PIS wt F and PIS wt R; PIS var-1 F and PIS var-1 R; and PIS var-2 F and PIS var-2 R, and a probe;
SRY F:
5′-GGGGCAATCGTATGCTTCT-3′,
shown as SEQ ID No. 1;
SRY R:
5′-CGGGTATTTGTCTCGGTGT-3′,
shown as SEQ ID No. 2;
PIS wt F:
5′-CTTGTGCCTCTTAGTCCTG-3′,
shown as SEQ ID No. 5;
PIS wt R:
5′-ACATAGAATGCCCTGGTG-3′,
shown as SEQ ID No. 6;
PIS var-1 F:
5′-ATTTGCTGGCGTATTGAGTG-3′,
shown as SEQ ID No. 7;
PIS var-1 R:
5′-AAGACGGTAGGTTCTGTGGG-3′,
shown as SEQ ID No. 8;
PIS var-2 F:
5′-TCATAGGCCATAGCTAAATGGT-3′,
shown as SEQ ID No. 9;
PIS var-2 R:
5′-GAGACAGGCTGAATGTGCAA-3′,
shown as SEQ ID No. 10;
the probe:
5′-TCATAGGCCATAGCTAAATGGTACTTAGA
GAAATTTTTATAGATTTAAATACATTCATCAG
ACTACAAATGAAGTGTAGGCATTAAAATTAAG
AAACTTGAAGAATGTGTATGTACTTCCAAAGC
AAAAATAAGAAAAATAAGGGTATAACTCGCTT
CTAGGGACTCAGGGGCAGTCTAATTTTCAAGC
ACTGAGAAGCCCAGATTAGTCTAATCAGTGCA
AGCCTGTCTTTCAGTCCATTTAAAGATCCTTT
GCACATTCAGCCTGTCTC-3′,
shown as SEQ ID No. 11.

2. The primer and probe group for detecting PIS genotype of goats according to claim 1, further comprising a specific primer pair of GAPDH F and GAPDH R;
GAPDH F:
5′-GTTTGTGATGGGCGTGAA-3′,
shown as SEQ ID No. 3;
GAPDH R:
5′-CTGTTGCTGTAGCCGAAT-4′,
shown as SEQ ID No. 4.

3. A kit based on the detection primer and probe group of claim 1.

4. An application of the detection primer and probe group of claim 1 in livestock breeding.

5. A PIS genotype detection method for goats based on the detection primer and probe group of claim 2, comprising the following steps:
S1 conducting primer design: comprising genetic sex determination primers and PIS genotype identification primers;
S2 conducting genetic sex and PIS genotype identification; and
S3 preparing a probe and conducting Southern hybridization detection.

6. The PIS genotype detection method for goats according to claim 5, wherein the step (2) comprises: extraction of sample genome DNA; PCR amplification of the genetic sex determination primers SRY-F/R and GAPDH-F/R; PCR amplification of the PIS genotype identification primers PIS wt F/R and PIS var-1 F/R; a 25 μL system is adopted during amplification and comprises 22 μL of 1.1×Golden Star Super PCR Mix, 1 μL of template DNA, 1 μL of forward primers and 1 μL of reverse primers; and reaction procedures are as follows: conducting pre-denaturation at 95° C. for 5 min, conducting denaturation at 95° C. for 30 s, annealing for 30 s, extending at 72° C. for 30 s, conducting cycles for 34 times, conducting renaturation at 72° C. for 5 min, and preserving the product at 4° C.

7. The PIS genotype detection method for goats according to claim 5, wherein the step (3) comprises: sample extraction; PIS var-2 F/R primer synthesis; recovery and purification of a target fragment; clone sequencing; extraction of a recombinant plasmid; preparation of a probe; labeling of the probe; digestion of genome DNA; and electrophoresis, membrane transfer, hybridization, membrane cleaning and signal detection on a digestion product;
wherein PIS var-2 F/R primer synthesis: a 25 μL system is adopted and comprises 22 μL of 1.1×Golden Star Super PCR Mix, 1 μL of template DNA, 1 μL of forward primers and 1 μL of reverse primers; and PCR reaction procedures are as follows: conducting pre-denaturation at 95° C. for 5 min, conducting denaturation at 95° C. for 30 s, annealing at 60° C. for 30 s, extending at 72° C. for 30 s, conducting cycles for 34 times, conducting renaturation at 72° C. for 5 min, and preserving the product at 4° C.;
clone sequencing: the carrier is a pGM-T carrier; a reaction system comprises 1 μL of 10×T4 DNA Ligation Buffer, 1 μL of 3 U/μL T4 DNA Ligase, 1 μL of a 50 ng/μL pGM-T carrier and 7 μL of a target PCR fragment; and overnight ligation is conducted at 16° C.;
preparation of the probe: the probe is synthesized through PCR by taking a recombinant plasmid as a template; labeled mononucleotide is doped into the newly synthesized DNA strands in the presence of Taq polymerase; a 50 μL system is adopted during PCR amplification and comprises 5 μL of plus Mg²⁺10×buffer, 5 μL of a dUTP labeled mixture, 1 μL of forward primers, 1 μL of reverse primers, 1 μL of the Taq polymerase, 1 μL of the recombinant plasmid and 36 μL of ddH₂O; and PCR reaction procedures are as follows: conducting pre-denaturation at 95° C. for 5 min, conducting denaturation at 95° C. for 30 s, annealing at 55° C. for 30 s, extending at 72° C. for 20 s, conducting cycles for 35 times, conducting renaturation at 72° C. for 7 min, and preserving the product at 4° C.;
digestion of genome DNA: a 800 μL system is adopted and comprises 60 μL of 10×buffer, 15 μL of BgI II, 15 μL of EcoR I, 30 μL of genome DNA and 680 μL of ddH₂O.

8. The PIS genotype detection method for goats according to claim 5, wherein detection standards are as follows:
Genetic Sex:
horned ewes: the amplification product has only one band of 579 bp;
horned rams: the amplification product has two bands of 579 bp and 379 bp simultaneously;
polled ewes: the amplification product has only one band of 579 bp;
polled rams: the amplification product has two bands of 579 bp and 379 bp simultaneously;
intersex goats: the amplification product has only one band of 579 bp; and the chromosome is XX;
PIS Typing:
wild-type homozygous horned goats: the amplification product has only one fragment of 214 bp;
deletion-mutation homozygous intersex goats: the amplification product has only one fragment of 680 bp, and there is a band during Southern hybridization detection;
dual-deletion homozygous polled rams: the amplification product has only one fragment of 680 bp, and there is a band during Southern hybridization detection;
deletion-mutation homozygous polled goats: the amplification product has fragments of 214 bp and 680 bp simultaneously, and there is a band during Southern hybridization detection.
